(19) United States
(12) Patent Application Publication
(10) Pub. No.: US 2006/0247249 A1
(43) Pub. Date: Nov. 2, 2006

(54) Carboximide derivatives as useful uro-selective alpha-1a adrenoceptor blockers

(76) Inventors: Mohammad Salman, Haryana Gurgaon (IN); Gyan Chand Yadav, Uttar Pradesh Ghaziabad (IN); Somesh Sharma, New Delhi (IN); Gobind Singh Kapkoti, Gurgaon (IN); Anita Chugh, New Delhi (IN); Jang Bahadur Gupta, Gurgaon (IN); Nitya Anand, Lucknow (IN)

Correspondence Address: RANBAXY INC., 600 COLLEGE ROAD EAST, SUITE 2100, PRINCETON, NJ, 08540, US

(21) Appl. No.: 10/510,668

(22) PCT Filed:
(86) PCT No.: PCT/IB02/01108

(51) Int. Cl. A61K 31/496; A61K 31/4178; C07D 405/02; C07D 409/02

(52) U.S. Cl. 514/252.13; 514/254.11; 514/389; 544/374; 544/375; 548/311.1

(57) ABSTRACT

Novel carboximide derivatives, which selectively inhibit binding to the α_{1A} adrenergic receptor, a receptor which has been shown to be important in the treatment of benign prostatic hyperplasia. The compounds of the present invention are potentially useful in the treatment of benign prostatic hyperplasia.

### FIELD OF THE INVENTION

This invention relates to certain novel carboximide derivatives which selectively inhibit binding to the α_{1A} adrenergic receptor, a receptor which has been shown to be important in the treatment of benign prostatic hyperplasia. The compounds of the present invention are potentially useful in the treatment of benign prostatic hyperplasia. This invention also relates to methods for synthesizing the novel compounds, pharmaceutical compositions containing the compounds, and method of treating benign prostatic hyperplasia using the compounds.

### BACKGROUND OF THE INVENTION

Benign prostatic hyperplasia (BPH), a nonmalignant enlargement of the prostate, is the most common benign tumor in men. Approximately 50% of all men older than 65 years have some degree of BPH and a third of these men have clinical symptoms consistent with bladder outlet obstruction (Hieble and Caine, Fed. Proc., 1986; 45:2601). Worldwide benign and malignant diseases of the prostate are responsible for more surgery than diseases of any other organ in men over the age of fifty.

It is generally accepted that there are two components of BPH, a static and a dynamic component. The static component is due to enlargement of the prostate gland, which may result in compression of the urethra and obstruction to the flow of urine from the bladder. The dynamic component is due to increased smooth muscle tone of the bladder neck and the prostate itself (which interferes with emptying of the bladder) and is regulated by alpha 1 adrenergic receptors (α₁-ARs). The medical treatments available for BPH address these components to varying degrees, and the therapeutic choices are expanding.

Surgical treatment options address the static component of BPH and include transurethral resection of the prostate (TURP), open prostatectomy, balloon dilatation, hyperthermia, stents and laser ablation. Although, TURP is the gold standard treatment for patients with BPH, approximately 20-25% of patients do not have a satisfactory long—term outcome (Lepor and Rigaud, J. Urol., 1990; 143:533). Postoperative urinary tract infection (5-10%), some degree of urinary incontinence (2-4%), as also reoperation (15-20%) (Wennberg et al., JAMA, 1987; 257:933) are some of the other risk factors involved.

Apart from surgical approaches, there are some drug therapies which address the static component of this condition. Finasteride (Proscar, Merck), is one such therapy which is indicated for the treatment of symptomatic BPH. This drug is a competitive inhibitor of the enzyme 5α-reductase which is responsible for the conversion of testosterone to dihydrotestosterone in the prostate gland (Gormley et al., N. Engl. J. Med., 1992; 327:1185). Dihydrotestosterone appears to be the major mitogen for prostate growth, and agents which inhibit 5-α-reductase reduce the size of the prostate and improve urine flow through the prostatic urethra. Although finasteride is a potent 5α-reductase inhibitor and causes a marked decrease in serum and tissue concentration of dihydrotestosterone, it is only moderately effective in treating symptomatic BPH (Oesterling, N. Engl. J. Med., 1995; 332:99). The effects of finasteride take 6-12 months to become evident and for many men the clinical improvement is minimal.

Due to the limited effectiveness of 5α-reductase inhibitors in terms of immediate symptomatic and urodynamic relief, other pharmacological approaches have been assessed in the clinical setting.

The dynamic component of BPH has been addressed by the use of adrenergic receptor blocking agents (α₁-AR blockers) which act by decreasing the smooth muscle tone within the prostate gland itself. α₁-adrenergic receptor antagonists appear to be much more effective and provide immediate subjective symptomatic improvements and are, therefore, the preferred modalities of treatment in the control of benign prostate hypertrophy. α₁-Adrenoceptors are also present in blood vessels and play an important role in the regulation of blood pressure. Thus, α₁-adrenoceptor antagonists are of particular importance as they were originally developed as antihypertensive agents and are likely also to have a beneficial affect on lipid dysfunction and insulin resistance, which are commonly associated with essential hypertension.

The use of α₁-AR antagonists in the treatment of BPH is related to their ability to decrease the tone of prostatic smooth muscle, leading to relief of the obstructive symptoms. Adrenergic receptors found throughout the body play a dominant role in the control of blood pressure, nasal congestion, prostate function and other processes (Harrison et al., Trends Pharmacol. Sci., 1991; 12:62). There are a number of cloned α₁-AR receptor subtypes: α_{1A}-AR, α_{1B}-AR and α_{1D}-AR (Bruno et al., Biochem. Biophys. Res. Commun., 1991; 179:1485; Forray et al., Mol. Pharmacol., 1994; 45:703; Hirasawa et al., Biochem. Biophys. Res. Commun., 1993; 195:902; Ramarao et al., J. Biol. Chem., 1992; 267:21936; Schwinn et al., JPET, 1995; 272:134; Weinberg et al., Biochem. Biophys. Res. Commun., 1994; 201:1296). A number of laboratories have characterized the α₁-ARS in human prostate by function, radioligand binding, and molecular biological techniques (Forray et al., Mol. Pharmacol. 1994; 45:703; Hatano et al., Br. J. Pharmacol, 1994; 113:723; Marshall et al., Br. J. Pharmacol. 1992; 112:59; Marshall et al., Br. J. Pharmacol., 1995; 115:781; Yamada et al., Life Sci., 1994; 54:1845). These studies provide evidence in support of the concept that the α_{1A}-AR subtype comprises the majority of α₁-ARS in human prostatic smooth muscle and mediates contraction in this tissue. These findings suggest that the development of a subtype-selective α_{1A}-AR antagonists might result in a therapeutically effective agent with reduced side effects for the treatment of BPH.

A variety of α₁-AR blockers (terazosin, prazosin, and doxazosin) have been investigated for the treatment of symptomatic bladder outlet obstruction due to BPH, with terazosin (Hytrin, Abbott) being the most extensively studied. Although the α₁-AR blockers are well tolerated, approximately 10-15% of patients develop a clinically adverse event. The undesirable effects of all members of this class are similar, with postural hypotension being the most commonly experienced side effect.

The α₁-AR blocking agents have a more rapid onset of action. However, their therapeutic effect, as measured by improvement in the symptom score and the peak urinary flow rate, is moderate. (Oesterling, N. Engl. J. Med., 1995; 332:99). The vascular side effects (e.g., postural hypertension, dizziness, headaches, etc.) associated with these drugs is due to lack of selectivity of action between prostatic and vascular α₁-adrenoceptors. Clearly, α₁-adrenoceptor antagonists which have inherently greater selectivity for prostatic α₁-adrenoceptors offer the potential of increased urodynamic benefits. This underscores the importance of the discovery of prostate-selective α₁-adrenoceptor antagonists which will confer urodynamic improvement without the side effects associated with existing drugs.

There are many description in the literature about the pharmacological activities associated with α,ω-dicarboximide derivatives. Eur. J. Med. Chem. Chemica Therapeutica; 1977; 12(2):173, J. Indian. Chem. Soc., 1978; LV:819; J. Indian Chem. Soc., 1979; LVI:1002 discuss the synthesis of these derivatives with CNS and antihypertensive activity. Other references like U.S. Pat. Nos. 4,524,206; 4,598,078; 4,567,180; 4,479,954; 5,183,819; 4,748,240; 4,892,943; 4,797,488; 4,804,751; 4,824,999; 4,957,913; 5,420,278; 5,330,762; 4,543,355 and PCT application Nos. WO 98/37893; WO 93/21179, also describe CNS and antihypertensive activity of these compounds. There is no mention of adrenoceptor blocking activity of these compounds and thus their usefulness in the treatment of BPH did not arise.

J. Med. Chem., 1983; 26:203 reports dopamine and α₁-adrenergic activity of some Buspirone analogues. EP 078800 discusses α₁-adrenergic receptor antagonistic activity of pyrimidinedione, pyrimidinetrione and triazinedione derivatives. These compounds, however, had low α₁-adrenergic blocking activity as compared to known α₁-antagonists.

The earlier synthesis of various 1-(4-arylpiperazin-1-yl)-3-(2-oxo-pyrrolidin-1-yl/piperidin-1-yl)alkanes and their usefulness as hypotensive and antischemic agents is disclosed in Indian Patent applications 496/DEU95, 500/DEU95 and 96/DEU96. These compounds had low α₁-adrenergic blocking activity (pKi˜6 as compared to >8 of the known α₁-antagonists such as prazosin), and practically no adrenoceptor sub-class selectivity for α_{1A} vs. α_{1B} or α_{1D} adrenoceptors. Further work showed that structural modification of these compounds from lactam to dioxo compounds, i.e., from 2-oxopyrrolidin to 2,5-dioxopyrrolidin and 2,6-dioxopiperidine, enhances the adrenoceptor blocking activity, and also greatly increases the selectivity for α_{1A} in comparison to α_{1B}-adrenoceptor blocking activity, an essential requirement for compounds to be good candidates for treatment of benign prostatic hyperplasia (BPH) disclosed in our U.S. Pat. Nos. 6,083,950 and 6,090,809 which are incorporated herein by reference.

### OBJECTS OF THE INVENTION

Recently, it has been demonstrated that the prostate tissue of higher species like man and dog has a predominant concentration of α_{1A}-adrenoceptor subtype. This makes it possible to develop agents with selective action against these pathological urodynamic states. The present invention is directed to the development of novel α₁-adrenoceptors and which would thus offer a viable selective relief for prostate hypertrophy as well as essential hypertension, without the side effects associated with known α_{1A}-AR antagonists.

The objective of the present invention is to provide novel carboximide derivatives that exhibit significantly greater α_{1A}-adrenergic blocking potency than available with known compounds in order to provide specific treatment for benign prostatic hyperplasia.

It is also an object of the invention to provide a process for synthesis of the novel compounds.

It is a further object of the invention to provide compositions containing the novel compounds which are useful in the treatment of benign prostatic hyperplasia.

### SUMMARY OF THE INVENTION

The above mentioned objectives are achieved by novel carboximide derivatives represented by Formula I below:

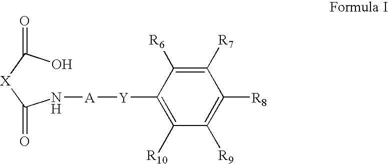

wherein X is selected from the group consisting of

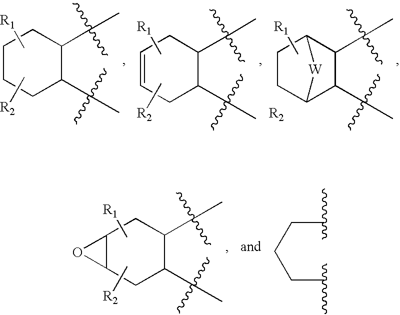

where the points of attachment are depicted by hashed bonds, and

where one point of attachment is bonded to the carbonyl adjacent to the nitrogen and the second point of attachment is bonded to the other carbonyl;

W is O, S, SO or SO₂

A is —(CH₂)m—,

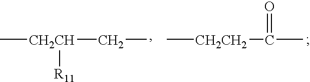

where m is one of the integers 2, 3 or 4;

R₁₁ is independently selected from H, F, Cl, Br, I, OH, straight or branched lower (C₁₋₆) alkyl, lower (C₁₋₆) alkoxy, lower (C₁₋₆) perhaloalkyl and lower (C₁₋₆) perhaloalkoxy;

Y is selected from the group consisting of

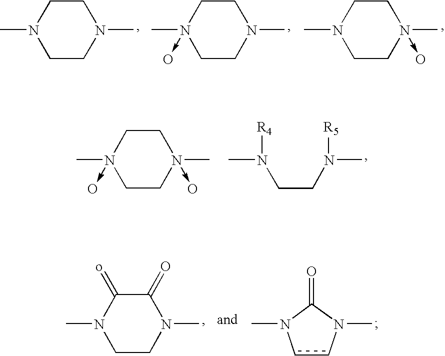

R₁ and R₂ are independently selected from H, OH, CN, NO₂, Cl, F, Br, I, OR₃, COR₃, OCOR₃, COOR₃, NH₂, N(R₄, R₁₅), lower (C₁₋₄)alkyl, lower (C₁₋₄)alkoxy, lower (C₁₋₄)alkylthio, lower (C₁₋₄) perhaloalkyl, lower (C₁₋₄) perhaloalkoxy, lower (C₁₋₄) alkoxy substituted with one or more of F, Cl, Br, I, OH, or OR₃, optionally substituted group selected from aryl, aryloxyaralkyl, heterocyclyl or heteroaryl and said substituents being H, F, Cl, Br, I, OH, OR₃, lower (C₁₋₄)alkyl, lower (C₁₋₄)alkyl substitued with one or more of F, Cl, Br, I, OH or OR₃, wherein R₃ is selected from the group consisting of H, straight or branched C₁-C₆ alkyl and perhaloalkyl; R₄ and R₅ are independently selected from the group consisting of H, CHO, substituted or unsubstituted lower (C₁₋₄)alkyl, lower (C₁₋₄)alkoxy, COR₃, COOR₃, CH₂CH(OR₃)₂, CH₂COOR₃, CH₂CHO and (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄)alkyl optionally substituted with one or more halogens, lower (C₁₋₄)alkoxy optionally substituted with one or more halogens, (C₃₋₆)cycloalkoxy, NH₂, N-lower (C₁₋₄)alkylamino, N,N-di-lower (C₁-C₄)alkylamino, N-lower (C₁-C₄) alkyl amino carbonyl, hydroxy substituted with aromatic or non-aromatic five or six membered ring, phenyl and phenyl substituted by Cl, F, Br, I, NO₂, NH₂, (C₁₋₄)alkyl or (C₁₋₄)alkoxy, (C₁₋₄) perhaloalkyl, (C₁₋₄) perhaloalkoxy wherein a broken line (----) is a single bond or no bond.

The present invention also provides pharmaceutical compositions for the treatment of benign prostatic hyperplasia. These compositions comprise an effective amount of at least one of the above compounds of Formula I and/or an effective amount of at least one physiologically acceptable acid addition salt thereof, with a pharmaceutically acceptable carrier and optionally included excipients.

An illustrative list of particular compounds of the invention is given below:
1-Carboxycyclohex-4-ene-2-[N-{3-(2-ethoxyphenyl)piperazin-1-yl}propyl]carboxamide, (Compound No. 1)
1-Carboxy cyclohex-4-ene-2-[N-{3-(2-isopropoxyphenyl)piperazin-1-yl}propyl]carboxamide, (Compound No. 2)
1-Carboxy cyclohex-4-ene-2-[N-{3-(2-methoxyphenyl)piperazin-1-yl}-2-hydroxypropyl]carboxamide, (Compound No. 3)
1-Carboxy cyclohex-4-ene-2-[N-{3-(2-hydroxyphenyl)piperazin-1-yl}-2-hydroxypropyl]carboxamide, (Compound No. 4)
1-Carboxy cyclohex-4-ene-2-[N-{3-(2-isopropoxyphenyl)piperazin-1-yl}-2-hydroxypropyl]carboxamide, (Compound No. 5)
1-Carboxy cyclohex-4-ene-2-[N-{3-(2-ethoxyphenyl)piperazin-1yl}-2-hydroxyphenyl]carboxamide, (Compound No. 6)
5-[N-{3-(2-hydroxyphenyl)piperazin-1-yl}]-1-aminopropyl-5-oxo-pentan-1-oic acid, (Compound No. 7)
1-Carboxy cyclohex-4-ene-2-[N-{3-(2-hydroxyphenyl)piperazin-1-yl}propyl]carboxamide, (Compound No. 8)
5-[N-{3-(2-Isopropoxyphenyl)piperazin-1-yl}-1-aminopropyl]-5-oxo-pentan-1-oic acid, (Compound No. 9)
Methyl-5-[N-{3-(2-methoxyphenyl)piperazin-1-yl}-1-aminopropyl]-5-oxo-pentanoate hydrochloride, (Compound No. 10)
1-Carboxymethylcyclohex-4-ene-2-[N-{3-(2-isopropoxyphenyl)piperazin-1-yl}-propyl]carboxamide hydrochloride, (Compound No. 11)
5-[N-{3-(2-Methoxyphenyl)piperazin-1-yl}]-2-hydroxypropylamino-5-oxo-pentan-1-oic acid, (Compound No. 12)

Pharmaceutically acceptable, non-toxic acid addition salts of the compounds of the present invention having the utility of the free bases of Formula I, may be formed with inorganic or organic acids, by methods well known in the art and may be used in place of the free bases. Representative examples of suitable acids for formation of such acid addition salts are malic, fumaric, benzoic, ascorbic, pamoic, succinic, bismethylene salicylic, methanesulfonic, ethane disulfonic, acetic, propionic, tartaric, salicylic, citric, gluconic, aspartic, stearic, palmitic, itaconic, glycolic, p-aminobenzoic, glutamic, benzenesulfamic, phosphoric, hydrobromic, sulfuric, cyclohexylsulfamic, hydrochloric, and nitric acids.

The present invention also includes within its scope prodrugs of the compounds of Formula I. In general, such prodrugs will be functional derivatives of these compounds which readily get converted in vivo into the defined compounds. Conventional procedures for the selection and preparation of suitable prodrugs are known.

The invention also includes the enantiomers, diastereomers, N-oxides, polymorphs, pharmaceutically acceptable salts and pharmaceutically acceptable solvates of these compounds, as well as metabolites having the same type of activity. The invention further includes pharmaceutical compositions comprising the molecules of Formula I, or prodrugs, metabolites, enantiomers, diastereomers, N-oxides, polymorphs, solvates or pharmaceutically acceptable salts thereof, in combination with a pharmaceutically acceptable carrier and optionally included excipients.

In yet another aspect, the invention is directed to methods for selectively blocking α_{1A} receptors by delivering in the environment of said receptors, e.g., to the extracellular medium (or by administering to a mammal possessing said receptors), an effective amount of the compounds of the invention.

### DETAILED DESCRIPTION OF THE INVENTION

In order to achieve the above mentioned objects and in accordance with the purpose of the invention as embodied and broadly described herein, there is provided a process for the synthesis of compounds of Formula I, as shown in Scheme I

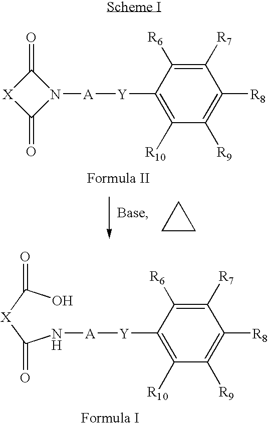

wherein X is selected from the group consisting of

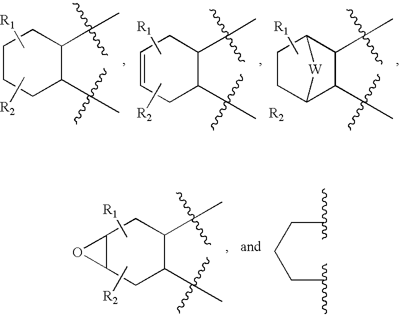

where the points of attachment are depicted by hashed bonds, and

where one point of attachment is bonded to the carbonyl adjacent to the nitrogen and the second point of attachment is bonded to the other carbonyl;

W is O, S, SO or SO₂

A is —(CH₂)m—,

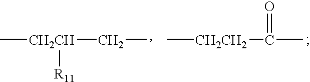

where m is one of the integers 2, 3 or 4;

R₁₁ is independently selected from H, F, Cl, Br, I, OH, straight or branched lower (C₁₋₆) alkyl, lower (C₁₋₆) alkoxy, lower (C₁₋₆) perhaloalkyl and lower (C₁₋₆) perhaloalkoxy;

Y is selected from the group consisting of

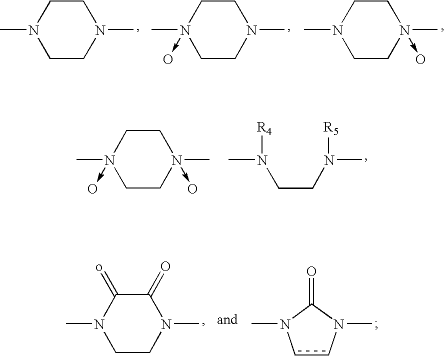

R₁ and R₂ are independently selected from H, OH, CN, NO₂, Cl, F, Br, I, OR₃, COR₃, OCOR₃, COOR₃, NH₂, N(R₄, R₅), lower (C₁₋₄)alkyl, lower (C₁₋₄)alkoxy, lower (C₁₋₄)alkylthio, lower (C₁₋₄) perhaloalkyl, lower (C₁₋₄) perhaloalkoxy, lower (C₁₋₄) alkoxy substituted with one or more of F, Cl, Br, I, OH, or OR₃, optionally substituted group selected from aryl, aryloxyaralkyl, heterocyclyl or heteroaryl and said substituents being H, F, Cl, Br, I, OH, OR₃, lower (C₁₋₄)alkyl, lower (C₁₋₄)alkyl substitued with one or more of F, Cl, Br, I, OH or OR₃, wherein R₃ is selected from the group consisting of H, straight or branched C₁-C₆ alkyl and perhaloalkyl; R₄ and R₅ are independently selected from the group consisting of H, CHO, substituted or unsubstituted lower (C₁₋₄)alkyl, lower (C₁₋₄)alkoxy, COR₃, COOR₃, CH₂CH(OR₃)₂, CH₂COOR₃, CH₂CHO and (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄)alkyl optionally substituted with one or more halogens, lower (C₁₋₄)alkoxy optionally substituted with one or more halogens, (C₃₋₆)cycloalkoxy, NH₂, N-lower (C₁₋₄)alkylamino, N,N-di-lower (C₁-C₄)alkylamino, N-lower (C₁-C₄) alkyl amino carbonyl, hydroxy substituted with aromatic or non-aromatic five or six membered ring, phenyl and phenyl substituted by Cl, F, Br, I, NO₂, NH₂, (C₁₋₄)alkyl or (C₁₋₄)alkoxy, (C₁₋₄) perhaloalkyl, (C₁₋₄) perhaloalkoxy wherein a broken line (----) is a single bond or no bond.

The starting materials of Scheme I may be suitably adapted to produce the more specific compounds of Formula I.

### SCHEME I

Scheme I shows the synthesis of the compounds of Formula I wherein X, Y, A, R₆, R₇, R₈, R₉ and R₁₀ are as defined above. The preparation comprises reacting α,ω-dicarboximides of Formula II with a suitable strong base, at a temperature ranging from 20-100° C. for a period varying between one to several hours to produce the corresponding compounds of Formula I. The suitable base is selected from the group consisting of sodium hydroxide and potassium hydroxide. More specifically, the hydrolysis of compound of Formula II is carried out in a solution of the base made in a polar solvent selected from the group consisting of water, methanol and ethanol. The preferable temperature conditions for the reaction are 90-100° C. The starting compound of Formula II can be prepared by the process as disclosed in our internal application number RLL-236WO filed concurrently herewith.

The invention is explained in detail in the example given below which is provided by way of illustration only and therefore should not be construed to limit the scope of the present invention.

### EXAMPLE

### Preparation of 1-carboxy cyclohex-4-ene-2-[N-{3-(2-ethoxyphenyl)piperazin-1-yl}propyl]carboxamide (Compound No. 1).

2-[3-{4-(2-Ethoxyphenyl)piperazin-1-yl}propyl-3a,4,7,7a-tetrahydro-1H-isoindole-1,3(2H)-dione hydrochloride (0.5 g, 1.15 mmol) was dissolved in aqueous sodium hydroxide solution (11.5 ml, 0.2N) and heated to reflux for about 2 hours. After the reaction was over, the pH of the reaction was adjusted to about 7 using glacial acetic acid and extracted with chloroform (2×15 ml). The solvent was concentrated under reduced pressure and the crude product was crystallized from chloroform and diethylether to afford the title product 0.13 g (25%), m.pt. 128-131° C.

MS m/z: 430.5 (MH⁺)

IR (KBr cm⁻¹): 1645.8 (C=0)

¹H NMR (300 MHz, TFA) δ:1.72-1.74 (3H,d), 2.59 (2H, br s), 2.80 (3H, br s), 2.93-2.99 (1H, d), 3.53 (1H, br s), 3.69 (1H, br s), 3.86-3.98 (m, 4H), 4.47-4.50 (6H, m), 4.75-4.79 (2H, m), 5.11-5.19 (1H, m), 6.00-6.13 (2H, br d), 7.39-7.49 (2H, m), 7.82-7.88 (2H, m)

The following compounds were prepared similarly:
1-Carboxy cyclohex-4-ene-2-[N-{3-(2-isopropoxyphenyl)piperazin-1-yl}propyl]carboxamide; m.p. 186-188° C., (Compound NO. 2)
1-Carboxy cyclohex-4-ene-2-[N-{3-(2-methoxyphenyl)piperazin-1-yl}-2-hydroxypropyl]carboxamide; m.p. 140-143° C., (Compound No. 3)
1-Carboxy cyclohex-4-ene-2-[N-{3-(2-hydroxyphenyl)piperazin-1-yl}-2-hydroxypropyl]carboxamide; m.p. 124-127° C., (Compound No. 4)
1-Carboxy cyclohex-4-ene-2-[N-{3-(2-isopropoxyphenyl)piperazin-1-yl}-2-hydroxy propyl]carboxamide; m.p. 159-162° C., (Compound No. 5)
1-Carboxy cyclohex-4-ene-2-[N-{3-(2-ethoxyphenyl)piperazin-1yl}-2-hydroxyphenyl]carboxamide; m.p. 118-121° C., (Compound No. 6)
5-[N-{3-(2-hydroxyphenyl)piperazin-1-yl}]-1-aminopropyl-5-oxo-pentan-1-oic acid; m.p. 200-202° C., (Compound No. 7)
1-Carboxy cyclohex-4-ene-2-[N-{3-(2-hydroxyphenyl)piperazin-1-yl}propyl]carboxamide; m.p. 165-170° C., (Compound No. 8)
5-[N-{3-(2-Isopropoxyphenyl)piperazin-1-yl}-1-aminopropyl]-5-oxo-pentan-1-oic acid; m.p. 121-125° C., (Compound No. 9)
Methyl-5-[N-{3-(2-methoxyphenyl)piperazin-1-yl}-1-aminopropyl]-5-oxo-pentanoate hydrochloride; m.p. 191-194° C., (Compound No. 10)
1-Carboxymethylcyclohex-4-ene-2-[N-{3-(2-isopropoxyphenyl)piperazin-1-yl}-propyl]carboxamide hydrochloride, (Compound No. 11)
5-[N-{3-(2-Methoxyphenyl)piperazin-1-yl}]-2-hydroxypropylamino-5-oxo-pentan-1-oic acid; m.p. 140-144° C., (Compound No. 12

### Pharmacological Testing Results

Receptor Binding Assays

Receptor binding assays were performed using native α-adrenoceptors. The affinity of different compounds for α_{1A} and α_{1B} adrenoceptor subtypes was evaluated by studying their ability to displace specific [³H] prazosin binding from the membranes of rat submaxillary and liver respectively (Michel et al, Br J. Pharmacol.; 1989; 98:883). The binding assays were performed according to U'Prichard et al. (Eur J Pharmacol., 1978; 50:87) with minor modifications.

Submaxillary glands were isolated immediately after sacrifice. The liver was perfused with buffer (Tris HCl 50 mM, NaCl 100 mM, 10 mM EDTA pH 7.4). The tissues were homogenised in 10 volumes of buffer (Tris HCl 50 mM, NaCl 100 mM, 10 mM EDTA pH 7.4). The homogenate was filtered through two layers of wet gauge and filtrate was centrifuged at 500 g for 10 min. The supernatant was subsequently centrifuged at 40,000 g for 45 min. The pellet thus obtained was resuspended in the same volume of assay buffer (Tris HCl 50 mM, 5 mM EDTA pH 7.4) and were stored at −70° C. until the time of assay.

The membrane homogenates (150-250 μg protein) were incubated in 250 μl of assay buffer (Tris HCl 50 mM, EDTA 5 mM, pH 7.4) at 24-25° C. for 1 hour. Non specific binding was determined in the presence of 300 nM prazosin. The incubation was terminated by vacuum filtration over GF/B fibre filters. The filters were then washed with ice cold 50 mM Tris HCl buffer (pH 7.4). The filtermats were dried and bound radioacivity retained on filters was counted. The IC₅₀ and Kd were estimated by using the non linear curve fitting program using G Pad Prism software. The value of inhibition constant Ki was calculated from competitive binding studies by using Cheng & Prusoff equation (Cheng & Prusoff, Biochem Pharmacol, 1973,22: 3099), Ki=IC₅₀/(1+L/Kd) where L is the concentration of [³H] prazosin used in the particular experiment (Table I).

In Vitro Functional Studies

In order to study selectivity of action of these compounds towards different α-adrenoceptor subtypes, the ability of these compounds to antagonise aorta (α_{1D}), prostate (α_{1A}) and spleen (α_{1B}) was studied. Aorta, prostrate and spleen tissues were isolated from urethane anaesthetized (1.5 g/kg) male wister rats. Isolated tissues were mounted in organ bath containing Krebs Henseleit buffer of the following composition (mM): NaCl 118; KCl 4.7; CaCl₂ 2.5; MgSO₄ 7H₂O 1.2; NaHCO₃ 25; KH₂PO₄ 1.2; glucose 11.5. Buffer was maintained at 37° C. and aereated with a mixture of 95% O₂ and 5% CO₂. A resting tension of 2 g (aorta) or 1 g (spleen and prostate) was applied to tissues. Contractile response was monitored using a force displacement transducer and recorded on chart recorders. Tissues were allowed to equilibrate for 2 hours. At the end of equilibration period, concentration response curves to norepinephrine (aorta) and phenylepinephrine (spleen and prostate) were obtained in the absence and presence of tested compound (at concentration of 0.1, 1 and 10 μM). Antagonist affinity was calculated and expressed as pK_{B} values in Table II.

In Vivo Uroselectivity Study

In order to assess the uroselectivity in vivo, the effects of these compounds were studied on mean arterial pressure (MAP) and intraurethral pressure (IUP) in conscious beagle dogs as per the method of Brune et. al. (Pharmacol., 1996, 53:356). Briefly, male dogs were instrumented for chronic continuous measurement of arterial blood pressure by implanting a telemetry transmitter (TL11 M2-D70-PCT, Data Sci. International, St. Paul, Minn. USA) into the femoral artery, two weeks prior to the study. During the recovery period, the animal was acclimatized to stay in the sling restraint. On the day of testing, overnight fasted animal was placed in the sling restraint. A Swan-Ganz. Balloon tipped catheter was introduced into the urethra at the level of prostate and the balloon was inflated (Brune. et. al. 1996). After recording the base line readings, effect of 16 μg/kg, phenylephrine (i.v.) on MAP and IUP was recorded. The response of phenylephrine to MAP and IUP were recorded at 0.5, 1, 2, 3, 4, 6, 9 and 24 hours after the oral administration of vehicle or the test drug. The changes in MAP were recorded on line using Dataquest Software (Data Sci. International. St. Paul, Minn. USA). The change in phenylephrine response on MAP and IUP administration after the test drug administration was calculated as percent change of that of control values. Area under curve was calculated and the ratio of the values for MAP and IUP was used for calculating the uroselectivity.

TABLE1
Radioligand Binding Studies:
Affinity of compounds for Alpha-1 Adrenoceptor Subtypes

| | | α_{1A} | α_{1B} | |
|---|---|---|---|---|
| S No | Compound No. | (Rat Submaxillary) | (Rat Liver) | α_{1B}/α_{1A} |
| 1 | 1 | 20 | >1000 | >50 |
| 2 | 2 | 19 | >1000 | >53 |
| 3 | 3 | >1000 | >1000 | |
| 4 | 4 | 1892 | 9743 | 5 |
| 5 | 5 | 21 | 1759 | 84 |
| 6 | 6 | 398 | 1239 | 3 |
| 7 | 7 | 12640 | 12970 | 1 |
| 8 | 8 | 593 | 5082 | 9 |
| 9 | 9 | 777 | 2097 | 3 |
| 10 | 10 | 139 | >1000 | >7 |
| 11 | 11 | 3.15 | 99 | 31 |
| 12 | 12 | >1000 | >10000 | 1 |

TABLE II
In Vitro Functional Assays

| | | α-Adrenoceptor Subtype | | | | |
|---|---|---|---|---|---|---|
| | Compound | (pK_{B}) | | | Selectivity | |
| S. No. | No. | α_{1A} | α_{1B} | α_{1D} | α_{1B}/α_{1A} | α_{1D}/α_{1A} |
| 1 | 1 | 8.22 | 8.16 | 7.24 | 1.15 | 9.5 |

While the present invention has been described in terms of its specific embodiments, certain modifications and equivalents will be apparent to those skilled in the art and are intended to be included within the scope of the present invention.

We claim:

1. A compound having the structure of Formula I, 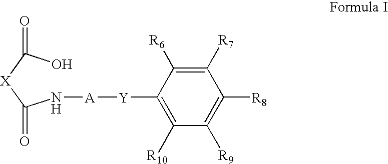
and its pharmaceutically acceptable salts, enantiomers, diastereomers, N-oxides, prodrugs, metabolities, polymorphs, or pharmaceutically acceptable solvates,
wherein
X is selected from the group consisting of 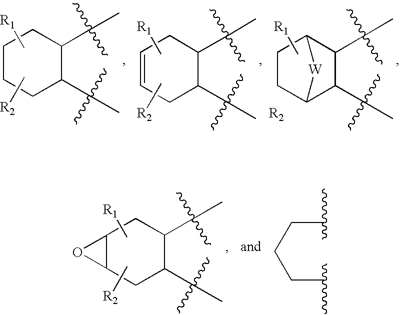
wherein the points of attachment are depicted by hashed bonds, and
wherein one point of attachment is bonded to the carbonyl adjacent to the nitrogen and the second point of attachment is bonded to the other carbonyl;
W is O, S, SO or SO₂;
A is —(CH₂)m—, 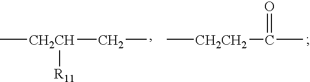
wherein m is one of the integers 2, 3 or 4;
R₁₁ is independently selected from H, F, Cl, Br, I, OH, straight or branched lower (C₁₋₆) alkyl, lower (C₁₋₆) alkoxy and lower (C₁₋₆) perhaloalkyl;
Y is selected from the group consisting of 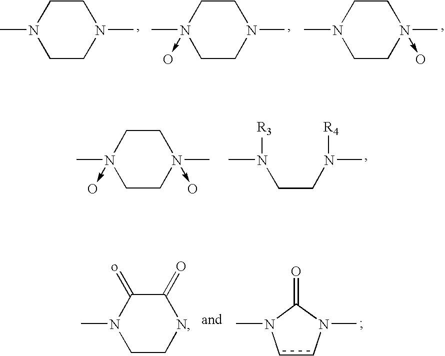
R₁ and R₂ are independently selected from H, OH, CN, NO₂, Cl, F, Br, I, OR₃,COR₃, OCOR₃, COOR₃, NH₂, N(R₄, R₅), lower (C₁₋₄)alkyl, lower (C₁₋₄) alkoxy, lower (C₁₋₄)alkylthio, lower (C₁₋₄)perhaloalkyl, lower (C₁₋₄) perhaloalkoxy, lower (C₁₋₄)alkoxy substituted with one or more of F, Cl, Br, I, OH, or OR₃, optionally substituted group selected from aryl, aryloxy, aralalkyl, heterocyclyl or heteroaryl and said substituents being H, F, Cl, Br, I, OH, OR₃, lower (C₁₋₄)alkyl, lower (C₁₋₄)alkyl substitued with one or more of F, Cl, Br, I, OH or OR₃, wherein R₃ is selected from the group consisting of H straight or branched C₁-C₆ alkyl and perhaloalkyl; R₄ and R₅ are independently selected from the group consisting of H, CHO, substituted or unsubstituted lower (C₁₋₄)alkyl, lower (C₁₋₄)alkoxy, COR₃, COOR₃, CH₂CH(OR₃)₂, CH₂COOR₃, CH₂CHO and (CH₂)₂OR₃ wherein R₃ is the same as defined above; R₆, R₇, R₈, R₉ and R₁₀ are independently selected from H, OH, CN, NO₂, Cl, F, Br, I, straight or branched lower (C₁₋₄)alkyl optionally substituted with one or more halogens, lower (C₁₋₄)alkoxy optionally substituted with one or more halogens, (C₃₋₈)cycloalkoxy, NH₂, N-lower(C₁₋₄)alkylamino, N,N-di-lower (C₁-C₄)alkylamino, N-lower alkyl(C₁-C₄)amino carbonyl, hydroxy substituted with aromatic or non-aromatic five or six membered ring, phenyl, phenyl substitued by Cl, F, Br, I, NO₂, NH₂, (C₁₋₄)alkyl or (C₁₋₄)alkoxy, (C₁₋₄)perhaloalkyl, (C₁₋₄)perhaloalkoxy wherein a broken line (....) is a single bond or no bond.

2. A compound selected from the group consisting of
1-Carboxy-cyclohex-4-ene-2-[N-{3-(2-ethoxyphenyl)piperazin-1-yl)propyl]carboxamide;
1-Carboxy-cyclohex-4-ene-2-[N-{3-(2-isopropoxyphenyl)piperazin-1-yl}propyl]carboxamide;
1-Carboxy cyclohex-4-ene-2-[N-{3-(2-methoxyphenyl)piperazin-1-yl}-2-hydroxypropyl]carboxamide;
1-Carboxy cyclohex-4-ene-2-[N-{3-(2-hydroxyphenyl)piperazin-1-yl}-2-hydroxypropyl]carboxamide;
1-Carboxy cyclohex-4-ene-2-[N-{3-(2-isopropoxyphenyl)piperazin-1-yl}-2-hydroxy propyl]carboxamide;
1-Carboxy cyclohex-4-ene-2-[N-{3-(2-ethoxyphenyl)piperazin-1yl}-2-hydroxyphenyl]carboxamide;
5-[N-{3-(2-hydroxyphenyl)piperazin-1-yl}]-1-aminopropyl-5-oxo-pentan-1-oic acid;
1-Carboxy cyclohex-4-ene-2-[N-{3-(2-hydroxyphenyl)piperazin-1-yl}propyl]carboxamide;
5-[N-{3-(2-Isopropoxyphenyl)piperazin-1-yl}-1-aminopropyl]-5-oxo-pentan-1-oic acid;
Methyl-5-[N-{3-(2-methoxyphenyl)piperazin-1-yl}-1-aminopropyl]-5-oxo-pentanoate hydrochloride;
1-Carboxymethylcyclohex-4-ene-2-[N-{3-(2-isopropoxyphenyl)piperazin-1-yl}-propyl]carboxamide hydrochloride;
5-[N-{3-(2-Methoxyphenyl)piperazin-1-yl}]-2-hydroxypropylamino-5-oxo-pentan-1-oic acid.

3. A method of selectively antagonizing α₁-adrenergic receptors in a mammal comprising administering to said mammal a therapeutically effective amount of a compound having the structure of Formula I: 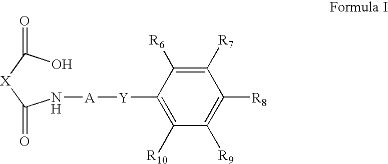
and its pharmaceutically acceptable salts, enantiomers, diastereomers, N-oxides, prodrugs, metabolities, polymorphs, or pharmaceutically acceptable solvates,
wherein
X is selected from the group consisting of 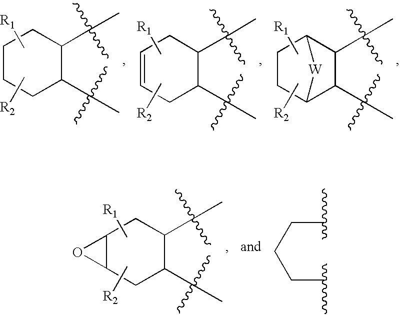
wherein the points of attachment are depicted by hashed bonds, and
wherein one point of attachment is bonded to the carbonyl adjacent to the nitrogen and the second point of attachment is bonded to the other carbonyl;
W is O, S, SO or SO₂;
A is —(CH₂)m—, 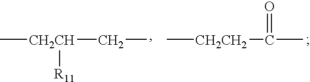
wherein m is one of the integers 2, 3 or 4;
R₁₁ is independently selected from H, F, Cl, Br, I, OH, straight or branched lower (C₁₋₆) alkyl, lower (C₁₋₆) alkoxy and lower (C₁₋₆) perhaloalkyl;
Y is selected from the group consisting of 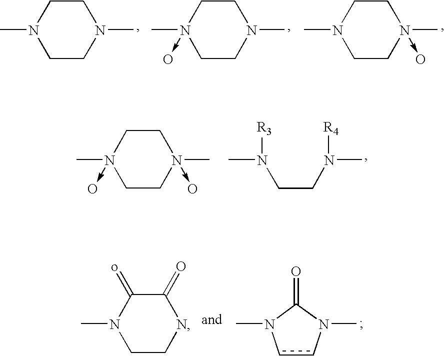
R₁ and R₂ are independently selected from H, OH, CN, NO₂, Cl, F. Br, I, OR₃,COR₃, OCOR₃, COOR₃, NH₂, N(R₄, R₁₅), lower (C₁₋₄)alkyl, lower (C₁₋₄) alkoxy, lower (C₁₋₄)alkylthio, lower (C₁₋₄)perhaloalkyl, lower (C₁₋₄)perhaloalkoxy, lower (C₁₋₄)alkoxy substituted with one or more of F, Cl, Br, I, OH, or OR₃, optionally substituted group selected from aryl, aryloxy, aralalkyl, heterocyclyl or heteroaryl and said substituents being H, F, Cl, Br, I, OH, OR₃, lower (C₁₋₄)alkyl, lower (C₁₋₄)alkyl substitued with one or more of F, Cl, Br, I, OH or OR₃, wherein R₃ is selected from the group consisting of H, straight or branched C₁-C₆ alkyl and perhaloalkyl; R₄ and R₅ are independently selected from the group consisting of H, CHO, substituted or unsubstituted lower (C₁₋₄)alkyl, lower (C₁₋₄)alkoxy, COR₃, COOR₃, CH₂CH(OR₃)₂, CH₂COOR₃, CH₂CHO and (CH₂)₂OR₃ wherein R₃ is the same as defined above; R₆, R₇, R₈, R₉ and R₁₀ are independently selected from H, OH, CN, NO₂, Cl, F, Br, I, straight or branched lower (C₁₋₄)alkyl optionally substituted with one or more halogens, lower (C₁₋₄)alkoxy optionally substituted with one or more halogens, (C₃₋₆)cycloalkoxy, NH₂, N-lower(C₁₋₄)alkylamino, N,N-di-lower (C₁-C₄)alkylamino, N-lower alkyl(C₁-C₄)amino carbonyl, hydroxy substituted with aromatic or non-aromatic five or six membered ring, phenyl, phenyl substitued by Cl, F, Br, I, NO₂, NH₂, (C₁₋₄)alkyl or (C₁₋₄)alkoxy, (C₁₋₄)perhaloalkyl, (C₁₋₄)perhaloalkoxy wherein a broken line (....) is a single bond or no bond.

4. A method for treating benign prostatic hyperplasia in a mammal comprising administering to said mammal a therapeutically effective amount of a compound having the structure of Formula I: 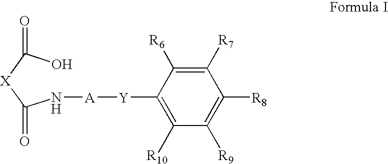
and its pharmaceutically acceptable salts, enantiomers, diastereomers, N-oxides, prodrugs, metabolities, polymorphs, or pharmaceutically acceptable solvates,
wherein
X is selected from the group consisting of 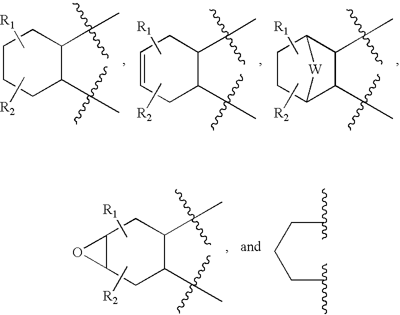
wherein the points of attachment are depicted by hashed bonds, and
wherein one point of attachment is bonded to the carbonyl adjacent to the nitrogen and the second point of attachment is bonded to the other carbonyl;
W is O, S, SO or SO₂;
A is —(CH₂)m—, 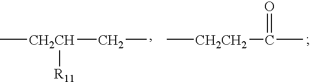
wherein m is one of the integers 2, 3 or 4;
R₁₁ is independently selected from H, F, Cl, Br, I, OH, straight or branched lower (C₁₋₆) alkyl, lower (C₁₋₆) alkoxy and lower (C₁₋₆) perhaloalkyl;
Y is selected from the group consisting of 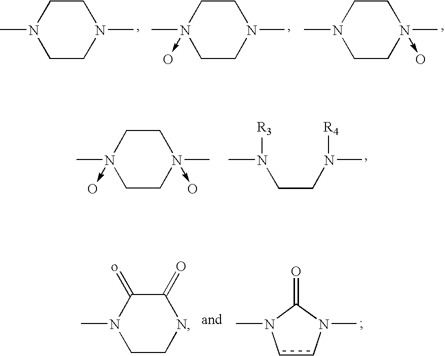
R₁ and R₂ are independently selected from H, OH, CN, NO₂, Cl, F, Br, I, OR₃,COR₃, OCOR₃, COOR₃, NH₂, N(R₄, R₅), lower (C₁₋₄)alkyl, lower (C₁₋₄) alkoxy, lower (C₁₋₄)alkylthio, lower (C₁₋₄)perhaloalkyl, lower (C₁₋₄)perhaloalkoxy, lower (C₁₋₄)alkoxy substituted with one or more of F, Cl, Br, I, OH, or OR₃, optionally substituted group selected from aryl, aryloxy, aralalkyl, heterocyclyl or heteroaryl and said substituents being H, F, Cl, Br, I, OH, OR₃, lower (C₁₋₄)alkyl, lower (C₁₋₄)alkyl substitued with one or more of F, Cl, Br, I, OH or OR₃, wherein R₃ is selected from the group consisting of H, straight or branched C₁-C₆ alkyl and perhaloalkyl; R₄ and R₅ are independently selected from the group consisting of H, CHO, substituted or unsubstituted lower (C₁₋₄)alkyl, lower (C₁₋₄)alkoxy, COR₃, COOR₃, CH₂CH(OR₃)₂, CH₂COOR₃, CH₂CHO and (CH₂)₂OR₃ wherein R₃ is the same as defined above; R₆, R₇, R₈, R₉ and R₁₀ are independently selected from H, OH, CN, NO₂, Cl, F, Br, I, straight or branched lower (C₁₋₄)alkyl optionally substituted with one or more halogens, lower (C₁₋₄)alkoxy optionally substituted with one or more halogens, (C₃₋₆)cycloalkoxy, NH₂, N-lower(C₁₋₄)alkylamino, N,N-di-lower (C₁-C₄)alkylamino, N-lower alkyl(C₁-C₄)amino carbonyl, hydroxy substituted with aromatic or non-aromatic five or six membered ring, phenyl, phenyl substitued by Cl, F, Br, I, NO₂, NH₂, (C₁₋₄)alkyl or (C₁₋₄)alkoxy, (C₁₋₄)perhaloalkyl, (C₁₋₄)perhaloalkoxy wherein a broken line (....) is a single bond or no bond.

5. A pharmaceutical composition comprising a therapeutically effective amount of a compound as defined in claim 1 or 2 and a pharmaceutical acceptable carrier.

6. A method of selectively antagonizing α₁-adrenergic receptors in a mammal comprising the step of administering to said mammal a therapeutically effective amount of the pharmaceutical composition according to claim 5.

7. A method for treating benign benign prostatic hyperplasia in a mammal comprising the step of administering to said mammal a therapeutically effective amount of the pharmaceutical composition according to claim 5.

8. A process for preparing a compound of Formula I 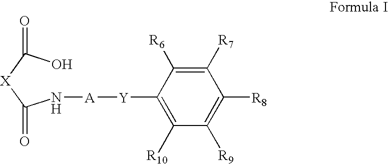
or its pharmaceutically acceptable salts, enantiomers, diastereomers, N-oxides, prodrgus, metabolities, polymorphs, and pharmaceutically acceptable solvates
wherein
X is selected from the group consisting of 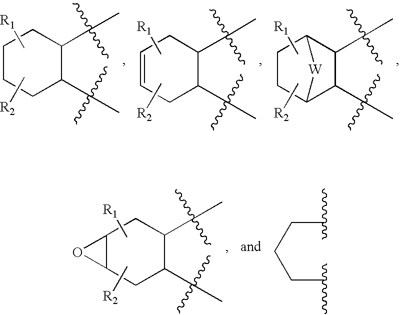
wherein the points of attachment are depicted by hashed bonds, and
wherein one point of attachment is bonded to the carbonyl adjacent to the nitrogen and the second point of attachment is bonded to the other carbonyl;
W is O, S, SO or SO₂;
A is —(CH₂)m—, 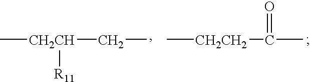
wherein m is one of the integers 2, 3 or 4;
R₁₁ is independently selected from H, F, Cl, Br, I, OH, straight or branched lower (C₁₋₆) alkyl, lower (C₁₋₆) alkoxy and lower (C₁₋₆) perhaloalkyl;
Y is selected from the group consisting of 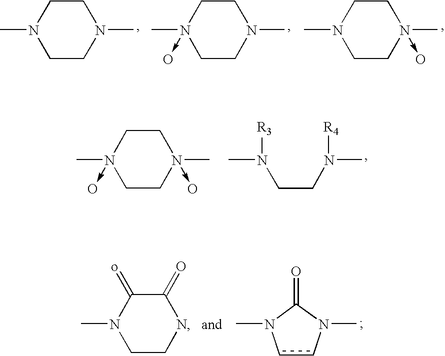
R₁ and R₂ are independently selected from H, OH, CN, NO₂, Cl, F, Br, I, OR₃,COR₃, OCOR₃, COOR₃, NH₂, N(R₄, R₅), lower (C₁₋₄)alkyl, lower (C₁₋₄) alkoxy, lower (C₁₋₄)alkylthio, lower (C₁₋₄)perhaloalkyl, lower (C₁₋₄) perhaloalkoxy, lower (C₁₋₄)alkoxy substituted with one or more of F, Cl, Br, I, OH, or OR₃, optionally substituted group selected from aryl, aryloxy, aralalkyl, heterocyclyl or heteroaryl and said substituents being H, F, Cl, Br, I, OH, OR₃, lower (C₁₋₄)alkyl, lower (C₁₋₄)alkyl substitued with one or more of F, Cl, Br, I, OH or OR₃, wherein R₃ is selected from the group consisting of H, straight or branched C₁-C₆ alkyl and perhaloalkyl; R₄ and R₅ are independently selected from the group consisting of H, CHO, substituted or unsubstituted lower (C₁₋₄)alkyl, lower (C₁₋₄)alkoxy, COR₃, COOR₃, CH₂CH(OR₃)₂, CH₂COOR₃, CH₂CHO and (CH₂)₂OR₃ wherein R₃ is the same as defined above; R₆, R₇, R₈, R₁₀ and R₁₀ are independently selected from H, OH, CN, NO₂, Cl, F, Br, I, straight or branched lower (C₁₋₄)alkyl optionally substituted with one or more halogens, lower (C₁₋₄)alkoxy optionally substituted with one or more halogens, (C₃₋₆)cycloalkoxy, 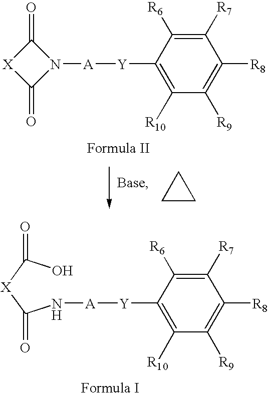
NH₂, N-lower(C₁₋₄)alkylamino, N,N-di-lower (C₁-C₄)alkylamino, N-lower alkyl(C₁-C₄)amino carbonyl, hydroxy substituted with aromatic or non-aromatic five or six membered ring, phenyl, phenyl substitued by Cl, F, Br, I, NO₂, NH₂, (C₁₋₄)alkyl or (C₁₋₄)alkoxy, (C₁₋₄)perhaloalkyl, (C₁₋₄)perhaloalkoxy wherein a broken line (....) is a single bond or no bond; which comprises reacting a compound Formula II with a suitable base in a suitable solvent to give the compound of Formula I as shown below:
where all symbols are as defined above.

9. The process of claim 8 wherein the base is selected from the group consisting of potassium hydroxide and sodium hydroxide.

10. The process of claim 8 wherein the suitable solvent is selected from the group consisting of water, methanol and ethanol.
